(19) United States
(12) Patent Application Publication
(10) Pub. No.: US 2021/0360955 A1
(43) Pub. Date: Nov. 25, 2021

(54) SYSTEM AND METHOD FOR FLAVOR ENCAPSULATION

(71) Applicant: Paragon Flavors, Inc., Princeton, NJ (US)

(72) Inventors: Matthew SILLICK, Montgomery, NJ (US); Christopher Mark GREGSON, Princeton, NJ (US)

(21) Appl. No.: 17/326,907

(22) Filed: May 21, 2021

Related U.S. Application Data
(63) Continuation-in-part of application No. 16/440,792, filed on Jun. 13, 2019.
(60) Provisional application No. 62/807,387, filed on Feb. 19, 2019.

(51) Int. Cl. A23L 27/00; A61K 31/05; A61K 9/48; A23L 27/12; A23L 33/115; A23L 11/70; A23L 7/25; A23L 33/105; A23L 33/125; A23P 10/30; A23P 10/28; A23L 2/39; A23L 2/04

(52) U.S. Cl. CPC A23L 27/72; A23V 2002/00; A61K 9/4833; A61K 9/4875; A23L 27/13; A23L 33/115; A23L 11/70; A23L 7/25; A23L 33/105; A23L 33/125; A23P 10/30; A23P 10/28; A23L 2/39; A23L 2/04; A61K 31/05

(57) ABSTRACT

An encapsulation method is disclosed for the capture of natural flavors within a clean-label derived from a malt comprising at least partially germinated whole grain seed, pulses, legumes, false grains or flour derived therefrom.

### CROSS REFERENCE TO RELATED APPLICATIONS SECTION

This application is a U.S. Non-Provisional Patent Application and Continuation-in-Part (CIP) Patent Application that claims priority to U.S. Non-Provisional patent application Ser. No. 16/440,792 filed on Jun. 13, 2019, which claims priority to the U.S. Provisional Application No. 62/807,387, filed Feb. 19, 2019, the entire contents of which are hereby incorporated by reference in their entirety.

### FIELD OF THE EMBODIMENTS

This invention pertains to an encapsulation system for capturing natural flavors, extracts and colors within a clean-label whole grain seed or flour carrier.

### BACKGROUND OF THE EMBODIMENTS

Processed foods are increasingly assailed as the main culprit for the obesity epidemic and ultimate cause of the increased incidence of adult type II diabetes in the United States. This perception of artificial food stuffs as being unhealthy has led to the consumer's widespread rejection of chemically modified starch (such as maltodextrin and octenyl succinate starch), polyols and gums in favor of organic, natural foods. According to a recent Clean Label Consumer Study, 78% of US consumers said it was important to recognize the ingredients used in the products they buy (an increase from 66% in 2011), and any ingredients that can't conceivably be found in the kitchen pantry fall outside consumers' definition of ‘clean label.’ The challenge for the food industry is how to provide the convenience of packaged foods made with recognizable kitchen cupboard ingredients without compromising taste.

Hence there is an ongoing need for carriers made from natural ingredients. The instant disclosure addresses this need by providing various powdered flavors using consumer preferred ingredients.

Examples of related art are described below:

U.S. Pat. No. 4,282,319 relates to a process for the preparation of hydrolyzed products from whole grain, and such derived products. The invention solves the problem of obtaining a protein and sugar containing product able to be filtrated whereby this is achieved by treating whole grain, such as wheat, maize, rye, barley, oat, and rice, with a proteolytic enzyme to transform water insoluble proteins into water soluble products, and further to treat the starch contents with an amylase free from other carbohydrate hydrolyzing enzymes to form water-soluble starch products, as mono and disaccharides, removing the bran fraction and removing water to obtain a dry, semi moist, or liquid but concentrated derived product. The product is to be added as a sweetening agent in food products as bread, drinks, and cereal products, whereby the bran obtained can be used in bread as fiber additive.

EP0078782B1 relates to a method for producing foodstuffs from whole cereal grain, and more particularly to a heat-technical wet processing method, where primarily the proteins of the respective cereal grain is so treated that its original nutritional value, related to the protein, is maintained intact for human consumption.

EP0922449B1 relates to a modified starch which is prepared by enzymatic hydrolysis of a starch molecule after the preparation of a starch derivative containing a hydrophobic group or both a hydrophobic and a hydrophilic group, particularly octenyl succinic anhydride starch hydrolyzed by beta-amylase or glucoamylase. Such modified starches are useful as encapsulating agents, particularly in systems where high load and retention of the active ingredient, low surface oil exposure, and excellent oxidation resistance is desired. The encapsulating agents are useful in a variety of applications including a tablet in which the starch allows for good compressibility and hardness.

U.S. Published Patent Application No. 2010/0196542 teaches the use of a maltodextrin and/or a glucose syrup obtained, by acid or enzymatic hydrolysis, from a leguminous starch having an amylose content comprised between 25% and 50%, expressed as dry weight relative to the dry weight of starch, for the encapsulation of organic compounds.

WO0238786A1 relates to an improved ethanol production process wherein the viscosity of liquefied mash and/or the thin stillage and/or condensate and/or syrup of evaporated thin stillage are reduced by addition of an effective amount of thinning enzyme selected from the group consisting of alpha-amylase, xylanase, xyloglucanase, cellulase, pectinase, or a mixture thereof.

WO2018/093285A1 pertains to the food industry, in particular to the brewing industry, and it can be used in the processing of grain in the process of making malt. More specifically, the disclosure describes a method of production of grain malt from cereal grain, which may involve: conditioning of the grain at moisture content of the grain in the range of 15-80% and for a time not longer than one day for moisture content in the indicated range; irrigation and/or soaking of the conditioned grain with an aqueous solution of a food enzyme having hydrolase activity

None of the art described above art addresses all the issues that the present disclosure does. For example, none of the art teaches the capture of natural and authentic extracts and colors within a clean-label wholegrain carrier comprising a malt of at least partially germinated whole grain seed or flour.

### SUMMARY OF THE EMBODIMENTS

In a first aspect, an encapsulate is disclosed comprising a clean label carrier dispersed with an active ingredient.

In certain embodiments of the first aspect, the clean label carrier does not contain a processed starch.

In certain embodiments of the first aspect, wherein the processed starch comprises a chemically modified starch, maltodextrin from refined starch, or cereal syrup solids (or tapioca or potato syrup solids).

In certain embodiments of the first aspect, the clean label carrier does not contain a gum extracted from plants, artificial flavors, artificial sweeteners or artificial preservatives.

In certain embodiments of the first aspect, the gum can be gum arabic, locust bean gum, gum ghatti, gum guaiacum, gum guar, or gum karaya.

In certain embodiments of the first aspect, the clean label carrier is mashed to a Brix value of at least 20.

In certain embodiments of the first aspect, the clean label carrier is mashed to a Brix value of between about 20 and about 50.

In certain embodiments of the first aspect, the clean label carrier is mashed to a refractive index greater than about 1.36 measured at 25° C.

In certain embodiments of the first aspect, the clean label carrier is mashed to a refractive index greater than 1.35.

In certain embodiments of the first aspect, the clean label carrier is mashed to a refractive index from about 1.35 to 1.45.

In certain embodiments of the first aspect, the clean label carrier is mashed to a refractive index from about 1.36 to 1.45.

In certain embodiments of the first aspect, the active ingredient comprises a natural extract, a fruit powder, a fruit or vegetable juice concentrate, a natural flavor, a pharmaceutical, a cannabidiol (CBD), an essence, a natural color, a juice extract, a biosynthetic color, cheese, nutraceutical and/or nutritional oil.

In certain embodiments of the first aspect, the active ingredient comprises a natural flavor. In certain embodiments of the first aspect, the natural flavor comprises a mint or lime oil.

In certain embodiments of the first aspect, the active ingredient comprises a natural color.

In certain embodiments of the first aspect, the natural color comprises beet juice.

In certain embodiments of the first aspect, the clean label carrier comprises a malt of at least partially germinated whole grain seed, pulses, legumes, false grains or flour thereof.

In certain embodiments of the first aspect, the clean label carrier comprises a malt of at least partially germinated legumes comprising chickpea, yellow pea, lentil, mung bean, green pea or flour derived therefrom.

In certain embodiments of the first aspect, the clean label carrier comprises a malt of rice flour.

In certain embodiments of the first aspect, the malt may comprise activated endogenous or exogenous glycosidases comprising alpha-amylases.

In certain embodiments, the clean label carrier comprises a malt of amaranth, barley, buckwheat, corn, millet, oats, quinoa, rice, rye, sorghum, teff, triticale, wheat or wild rice.

In certain embodiments of the first aspect, the clean label carrier further comprises carbohydrate sources from green bananas, peas, beans, roots, potato, tapioca, tapioca starch, rice starch, purple sweet potato, sweet potato or yam.

In certain embodiments of the first aspect, the clean label carrier is encapsulated by spray drying, low temperature spray drying, multistage spray drying, spray granulation, spray agglomeration, drying of starch-coated beadlets, fluidized bed drying, fluidized bed coating, melt injection, melt extrusion, drum drying, freeze drying, vacuum drying, prilling, belt drying, refractance window drying, mylar belt radiative drying or any combination thereof.

In a second aspect, a foodstuff is disclosed comprising an encapsulate of any of the preceding embodiments of the first aspect. The foodstuff can be a snack, a confection, dry beverage, dry doughs such as cake, cookie or bread mixes, stock cubes, crackers or tortilla, bakery mix, pancake mix, breakfast cereals, bars, protein smoothies, nutritional supplement, instant sauce mixes, spice rubs and marinades, stuffing, coffee/tea or meat and meat-alternatives.

In a third aspect, a method is disclosed for making an encapsulate comprising mashing a malt of a whole grain seed, pulses, legumes, false grains or flour thereof, wherein the mash has a Brix value of at least about 20; optionally filtering the mash, dispersing the mash with an active ingredient and drying the mash dispersion.

In certain embodiments of the third aspect, the mash has a ratio of the Brix value relative to the amount of flour solids present of at least about 0.5:1. In certain embodiments of the third aspect, the mash has a ratio of the Brix value relative to the amount of flour solids present of about 0.5:1 to 1:1.

In certain embodiments of the third aspect, the encapsulated mash dispersion is agglomerated, roller compacted, extrusion pelletized or coated.

In certain embodiments of the third aspect, the encapsulate does not contain processed starch, a gum extracted from plants, artificial flavors, artificial sweeteners or preservatives.

In certain embodiments of the third aspect, the encapsulate does not contain processed starch, a gum extracted from plants, artificial flavors, artificial sweeteners or artificial preservatives.

In certain embodiments of the first aspect, the active ingredient comprises a natural extract, a fruit or vegetable juice concentrate, a natural flavor, a pharmaceutical, a cannabidiol (CBD), an essence, a natural color, a juice extract, a biosynthetic color, cheese, nutraceutical and/or nutritional oil or any combination thereof.

In certain embodiments of the first aspect, the malt comprises a malt of rice flour.

### BRIEF DESCRIPTION OF THE DRAWINGS

FIG. 1 shows an exemplary depiction of flavor oil droplets emulsified in a carbohydrate matrix.

### DESCRIPTION OF THE PREFERRED EMBODIMENTS

The preferred embodiments of the present invention will now be described. Such embodiments are provided by way of explanation of the present invention, which is not intended to be limited thereto. Those of ordinary skill in the art may appreciate upon reading the present specification and viewing the present drawings that various modifications and variations can be made thereto.

### Definitions

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Common encapsulation carriers in the field are often made of highly refined, processed ingredients including, but not limited to, starch hydrolysates such as maltodextrins (CAS #9050-36-6), cyclodextrin (CAS #12619-70-4), syrups (CAS #8029-43-4) or dextrins (CAS #9004-53-9). Materials used for encapsulation are reviewed in detail by Christine Wandrey, Artur Bartkowiak, and Stephen E. Harding, Chapter 3 of the book entitled “Encapsulation Technologies for Active Food” by N. J. Zuidam and V. A. Nedović (eds.), Springer Science+Business Media, LLC 2010, the content of which is incorporated by reference herein in its entirety.

While modified starches and maltodextrins from refined starches have been successfully used for coating and encapsulation, they are no longer accepted by the majority of health-conscious consumers who desire natural food products not processed ones. For this reason, the present encapsulation process does not use any “poorly accepted,” including processed starches, artificial sweeteners or artificial coloring. The present encapsulate is manufactured solely from a starting material comprising clean label ingredients such as whole grain seed or flour derived therefrom including, but not limited to, unsprouted corn, barley, wheat, rice, tapioca, green banana, pea, bean/legumes, potato, purple sweet potato, oat, amaranth, buckwheat, rye, yam, jicama, arrowroot, sorghum or sago.

“Clean label” refers is a consumer driven movement, demanding a return to real food and transparency through authenticity. The Clean Label Project™ uses data and science to reveal the true contents of America's best-selling consumer products. Products are tested in an accredited analytical chemistry laboratory for 130 harmful environmental and industrial contaminants and toxins. Results are published as Product Ratings on the gocleanlabel web site, the content of which is incorporated by reference herein in its entirety. Thus, the present encapsulate comprises natural, familiar, simple ingredients and excludes any artificial or processed ingredients as well as synthetic chemicals.

As used herein, a “clean label carrier” refers to an encapsulating material comprising a whole grain seed or flour, or derivative thereof, that is capable of capturing an active ingredient such as a natural flavor. Thus the “clean label carrier” does not include any artificial or refined ingredients including modified starches, maltodextrins, cyclodextrin, syrups, artificial sweeteners, artificial coloring or synthetic chemicals.

As used herein, the term “processed starch” refers to starch treated physically (e.g. by heat), chemically and/or enzymatically.

In certain embodiments, the active ingredient comprises a natural extract, a fruit or vegetable juice concentrate, a natural flavor, a pharmaceutical, a cannabidiol (CBD), an essence, a natural color, a juice extract, a biosynthetic color, cheese, nutraceutical and/or nutritional oil or any combination thereof.

In certain embodiments, a processed starch can be a “starch derivative” that includes, but is not limited to (1) starch or starch components that have been isolated from their grain to remove protein and bran and then partly digested with enzymes or partly hydrolyzed by heat and/or acids, e.g. maltodextrins; or (2) starch or starch components that are chemically modified, e.g. etherified or esterified starch, for better emulsification, solubility or digestibility.

In certain embodiments, the starting material of whole grain seed is dehusked, a process in which the seed's husk, pericarp and seed coat (testa) is removed by mechanical treatment prior to malting. An exemplary method of dehusking grains can be found in the U.S. Pat. No. 3,708,002, the content of which is incorporated by reference herein in its entirety.

A key feature of the present encapsulation method resides in the malting of whole grain seed, or flour, that requires an at least partial germination of the whole grain seed.

### Malting Step

Seeds comprise a storage of starch, proteins, and oils that are metabolized during germination to provide nutrients to the growing embryo. Germination typically is initiated by the adsorption of water and subsequent swelling of the seed in a process called imbibition. Provided favorable conditions for seed germination exist, this hydration activates endogenous hydrolytic enzymes, glycosidases, such as alpha-amylase, and proteases that then break down the stored food resources needed for the ensuing rapid cell division of the plant embryo. Thus, “germination” starts with imbibition, seed swelling, and enzyme activation and concludes with the emergence or “sprouting” of a seedling comprising a leaf and a root through the seed coat at which point photosynthesis kicks in to furnish the energy necessary for further growth of the plant.

In certain embodiments, once initiated by imbibition, germination can take approximately 1-3 weeks to complete depending on the species of plant.

Favorable conditions for seed germination require optimal oxygen, humidity, light or darkness and temperature. The optimal temperature for germination of most seeds is in the range of just above 0° C. to about 41° C., more preferably 21° C. to 35° C. By the term “just above 0° C.” is meant a temperature at which (at atmospheric pressure) water is liquid.

In certain embodiments, the term “at least partially sprouted whole grain seed” refers to the state of seed germination after about ½, 1, 2, 3, 4, 5, 6, 7, 8, 9, 10, 11, 12, 13, 14, 15, 16, 17, 18, 19, 20 or 21 days from the initiation of imbibition.

In certain embodiments, the term “at least partially sprouted whole grain seed” refers to a population of seeds in which about 5%, 10, 15, 20%, 25, 30%, 35%, 40%, 45%, 50%, 55%, 60%, 65%, 70%, 75%, 80%, 85%, 90%, 95% or 99% have a visible “sprouting” root, shoot or leaf.

In certain embodiments, the sprout to seed length ratio can be 0.25:1, 0.5:1, 0.75:1, 1:1, 2:1, 4:1 or 8:1.

In certain embodiments, germination of whole grain seed, e.g., barley, results in the conversion of the starchy endosperm into glucose by a concerted action of alpha- and beta-amylases, debranching enzyme and alpha-glucosidase (maltase).

In certain embodiments, the activation of α-amylase during germination hydrolyzes alpha-bonds in starch at random locations on the polymer chain, thereby making it a faster enzyme compared to other amylases. In certain embodiments, the end products can be maltose and glucose from amylopectin and maltose and maltotriose from amylose.

In certain embodiments, the enzymatic activity of endogenous glycosidases following imbibition can be measured using methods well known in the art.

For example, the Falling Number (FN), also referred to as the Hagberg Number, is an internationally standardized (ICC 107/1, ISO 3093-2004, AACC 56-81B, ASBC Barley 12-A) method of assessing the presence of glycosidase activity induced by the onset at least partial germination. The FN is determined by measuring the time required for a plunger to fall through a heated slurry of whole meal and water thus detecting changes in the viscosity of the whole_meal-water slurry due to the presence of carbohydrate-degrading enzymes such as amylases (Hagberg, S. Cereal Chemistry 37 (1960) 218; Perten, H. Cereal Chemistry 41 (1964) 127-140). In the absence of germination, the level of glycosidase activity is very low and the Falling Number has a high value. Conversely, a low falling number is an indicator for the presence of decreased viscosity as a result of increased glycosidase activity.

Alternative methods of measuring glycosidase activity include the Rapid ViscoAnalyzer (Ross, A. S.; Orth, R. A.; Wrigley, C. W. Proceedings of the Fourth International Symposium on Pre-Harvest Sprouting in Cereals, D. J. Mares (Ed.) Westview Press, Boulder, Colo., USA (1987) 577-583) and Near Infrared analysis (Czuchajowska, Z.; Pomeranz, Y. Preharvest Sprouting in Cereals 1992, M. K. Walker-Simmons and J. L. Ried (Eds.) American Association of Cereal Chemists, St Paul, Minn., USA (1992) 409-416). Near Infrared predictions of Falling Number are also of relatively low precision and can only discriminate relatively large differences in Falling Number (Osborne, B. G. Journal of the Science of Food and Agriculture, 35 (1984) 106-110; Czuchajowska, Z.; Pomeranz, Y.; Preharvest Sprouting in Cereals 1992, M. K. Walker-Simmons and J. L. Ried (Eds.) American Association of Cereal Chemists, St Paul, Minn., USA (1992) 409-416; Shashikumar, K.; Hazleton, J. L.; Ryu, G. H.; Walker, C. E. Cereal Foods World 38 (1993) 264-269).

In certain embodiments, alpha-amylase can be detected using an immunoassay (see, for example, U.S. Pat. No. 7,074,579, the content of which is incorporated by reference herein in its entirety).

In certain embodiments, alpha-amylase activity can be measured directly using a “Phadebas Amylase Test” (PAT) kit, commercially available from Phadebas AB. Phadebas is a synthetic biochemical substrate whose active component is DSM-P, microspheres in which a blue dye has been chemically bound. When the substrate is digested by the amylase enzyme in solution, it releases that blue dye at a rate proportional to the quantity and activity of the enzyme present.

Methods of measuring changes in alpha- and beta-amylase activities during seed germination of African finger millet can be found, for example, in Gimbi D M, Kitabatake N. Int J Food Sci Nutr. 2002 November; 53(6):481-8, the content of which is incorporated by reference herein in its entirety.

In certain embodiments, the malting process prevents fermentation and enhances enzyme build-up by using a series of washing steps in conjunction with the addition of antibiotic agents (e.g. tea tree oil) that inhibit yeast growth. A person skilled in the art would understand that the malting process requires careful adjustment of temperature, relative humidity and grain moisture to obtain optimal enzymatic activity. In certain embodiments, the amount of alcohol present in the malt or mash is less than 5%, 4%, 3%, 2% or 1%. In certain embodiments, the amount of alcohol present in the malt or mash is about 0%. In preferred embodiments, the amount of alcohol present in the malt or mash is 0%.

In certain embodiments, the grain can be placed in a wet mill before drying.

In certain embodiments, the germination process is halted by drying the partially germinated whole grain seed on a fluid bed drier or flash drier.

In certain embodiments, fast drying on a large surface area at low temperature preserves the malt's enzymatic activity.

In certain embodiments, the husk is not removed prior to the malting process.

In certain embodiments, addition of cysteine and/or washing steps can facilitate the release of endogenous enzymes within the malt.

In certain embodiments, water is added to unmalted flour and/or sprouted (malted) grain flour that is milled into a flour.

In certain embodiments, sugars and oligosaccharides such as maltose, sucrose, glucose, fructose, allulose, allose, trehalose, trehalulose, innulin, fructo-oligosaccharides, dextrins, dextran, alternan, phytoglycogen, polydextrin, beta-glucan, fruit juices, vegetable juices, fruit & vegetable puree and puree concentrates, xylitol, xylose, erythritol can be added to the malt prior to, during or after mashing.

An advantage of the aforementioned malting process is to provide a subtle malt flavor which can enhance the overall profile of the powdered flavor.

### Mashing Step

Soluble film-forming oligosaccharide molecules are formed by a mashing step. These constituents increase the refractive index of the mash dispersion. This is essential because such molecules enrobe and entrap active ingredients. Unmashed starches are viscous and do not enrobe.

In certain embodiments, during the mashing step, the endogenous enzymes present in the malt of the at least partially germinated whole grain seed convert the stored starch to produce a soluble film-forming oligosaccharide solution suitable for spray processing. In certain embodiments, the malt is milled into a flour prior to mashing.

In certain embodiments, the malt is mixed in the presence of water.

Optimal enzymatic activity can be achieved by adjusting the temperature, ion concentration, pH and dilution of the malt.

In certain embodiments, the malt flour is gradually added to the mash so as to avoid an increase in viscosity.

In certain embodiments, endo-enzymes, exo-enzymes, maltotetrose cleaving enzymes, thermostable enzymes, debranching enzymes such as pullulanase or other natural source of enzymes may be added to the mash as needed. In certain embodiments, the exogenous enzymes can be an alpha- or beta-amylase.

In certain embodiments, exogenous amylase can be added to supplement the endogenous amylase and achieve a total alpha amylase activity of 20 and 200 units/gram. Methods of measuring alpha-Amylase activity in white wheat flour are described, for example, in Mccleary et al. Journal of Aoac International vol. 85, No. 5, 2002, the content of which is incorporated by reference herein in its entirety.

For example, test mash samples are clarified by centrifugation or filtration and diluted in a salt/buffer solution. Aliquots of diluted extract are incubated with a substrate mixture under defined conditions of pH, temperature, and time. The substrate can be nonreducing end-blocked p-nitrophenyl maltoheptaoside (BPNPG7) in the presence of excess quantities of thermostable α-glucosidase. The blocking group in BPNPG7 prevents hydrolysis of this substrate by exo-acting enzymes such as amyloglucosidase, α-glucosidase, or β-amylase. When the substrate is cleaved by endo-acting α-amylase, the nitrophenyl oligosaccharide is immediately and completely hydrolyzed to p-nitrophenol and free glucose. The reaction is then terminated, and the phenolate color developed by the addition of an alkaline solution is measured at 400 nm. Results are expressed in Ceralpha units, 1 unit being defined as the amount of enzyme required to release (in the presence of excess α-glucosidase) 1 μmol p-nitrophenol per minute at 40° C.

In certain embodiments, a malt has an amylase activity of about 100 U/g of starch.

In certain examples, malted barley can have an alpha amylase activity of between 100 and 200 U/g of starch.

In certain examples, sprouted and malted rice flours can have an alpha amylase activity between about 5 and 50 U/g.

In certain examples, sprouted and malted rice flours can have an alpha amylase activity between about 5 and 100 U/g.

In certain examples, sprouted and malted rice flours can have an alpha amylase activity between about 5 and 150 U/g.

In certain examples, sprouted and malted rice flours can have an alpha amylase activity between about 5 and 250 U/g.

In certain examples, sprouted and malted rice flours can have an alpha amylase activity between about 5 and 500 U/g.

In certain examples, an “at least partially germinated whole grain seed or flour” can have an alpha amylase activity between about 10 and 500 U/g.

In certain examples, an “at least partially germinated whole grain seed or flour” can have an alpha amylase activity between about 15 and 50 U/g.

In certain examples, an “at least partially germinated whole grain seed or flour” can have an alpha amylase activity between about 20 and 50 U/g.

In certain examples, an “at least partially germinated whole grain seed or flour” can have an alpha amylase activity between about 25 and 50 U/g.

In certain examples, an “at least partially germinated whole grain seed or flour” can have an alpha amylase activity between about 30 and 50 U/g.

In certain examples, an “at least partially germinated whole grain seed or flour” can have an alpha amylase activity between about 35 and 50 U/g.

In certain examples, an “at least partially germinated whole grain seed or flour” can have an alpha amylase activity between about 40 and 50 U/g.

In certain examples, an “at least partially germinated whole grain seed or flour” can have an alpha amylase activity between about 45 and 50 U/g.

In certain examples, an “at least partially germinated whole grain seed or flour” can have an alpha amylase activity between about 10 and 45 U/g.

In certain examples, an “at least partially germinated whole grain seed or flour” can have an alpha amylase activity between about 10 and 40 U/g.

In certain examples, an “at least partially germinated whole grain seed or flour” can have an alpha amylase activity between about 10 and 35 U/g.

In certain examples, an “at least partially germinated whole grain seed or flour” can have an alpha amylase activity between about 10 and 30 U/g.

In certain examples, an “at least partially germinated whole grain seed or flour” can have an alpha amylase activity between about 10 and 25 U/g.

In certain examples, an “at least partially germinated whole grain seed or flour” can have an alpha amylase activity between about 10 and 20 U/g.

In certain examples, an “at least partially germinated whole grain seed or flour” can have an alpha amylase activity between about 10 and 15 U/g.

In certain examples, an “at least partially germinated whole grain seed or flour” can have an alpha amylase activity between about 15 and 45 U/g.

In certain examples, an “at least partially germinated whole grain seed or flour” can have an alpha amylase activity between about 20 and 40 U/g.

In certain examples, an “at least partially germinated whole grain seed or flour” can have an alpha amylase activity between about 25 and 35 U/g.

In certain examples, an “at least partially germinated whole grain seed or flour” can have an alpha amylase activity between about 25 and 30 U/g.

In certain examples, an “at least partially germinated whole grain seed or flour” can have an alpha amylase activity between about 30 and 35 U/g.

In certain examples, the “at least partially germinated whole grain seed or flour” comprises sprouted and malted rice flours having an amylase activity of between about 1 U/g and 200 U/g.

In certain embodiments, addition of exogenous amylase is not required provided the endogenous alpha amylase activity of the at least partially germinated whole grain seed or flour is more than 10 U/g.

The progress of the mashing step can be monitored using a refractometer to measure solubilization of polysaccharides in the mash.

The refractive index is a ratio of the speed of light in a medium relative to its speed in a vacuum. This change in speed from one medium to another is what causes light rays to bend. This is because as light travels through another medium other than a vacuum, the atoms of that medium constantly absorb and reemit the particles of light, slowing down the speed light travels at. Thus, the denser the liquid, the slower the light will travel through it, and the higher its reading will be on the refractometer.

The refractive index can be calculated using the equation below.

$${n}_{D}^{t} = \frac{speed of light in vacuum}{speed of light in the medium}$$

Many different scales are available that convert refractive index into a unit of measure that is more meaningful, i.e., Brix, specific gravity, Plato, etc.

An exemplary method of measuring a refractive index or Brix of a malt is taught, for example, in U.S. Pat. No. 9,448,166, the content of which is incorporated by reference herein in its entirety. Brix represents the physical/mathematical relationship between refractive index and the content of sucrose per weight in sucrose water solution. An exemplary conversion between refractive index resp density to Brix at 20° C. is depicted in TABLE I.

TABLE I
Refractive Index (1)

| Brix % | nD [symbol] |
|---|---|
| 0 | 1.33299 |
| 5 | 1.34026 |
| 10 | 1.34782 |
| 15 | 1.35568 |
| 20 | 1.36384 |
| 25 | 1.37233 |
| 30 | 1.38115 |
| 35 | 1.39032 |
| 40 | 1.399 [symbol] 6 |
| 45 | 1.40987 |
| 50 | 1.42009 |
| 55 | 1.43080 |
| 60 | 1.44193 |
| 65 | 1.45348 |
| 70 | 1.46546 |
| 75 | 1.477 [symbol] 7 |
| 80 | 1.49071 |
| 85 | 1.50398 |

Density (2)

| Brix % | SG [symbol] |
|---|---|
| 0 | 1.00000 |
| 5 | 1.00965 |
| 10 | 1.03998 |
| 15 | 1.06104 |
| 20 | 1.08287 |
| 25 | 1.10551 |
| 30 | 1.11898 |
| 35 | 1.15331 |
| 40 | 1.17853 |
| 45 | 1.204 [symbol] 7 |
| 50 | 1.23174 |
| 55 | 1.2597 [symbol] |
| 60 | 1.28873 |
| 65 | 1.31 [symbol] 6 |
| 70 | 1.34956 |
| 75 | 1.38141 |
| 80 | 1.41421 |
| 85 | 1.44794 |
| 90 | 1.48259 |
| 95 | 1.51814 |

(1) According to 16th Session of ICUMSA 1974
(2) Specific gravity according to 109 of NBS Circulor 440 [symbol]
indicates data missing or illegible when filed

In certain embodiments, the Brix value of the malt is at least 20, at least 25, at least 30, at least 35, at least 40, at least 50.

In certain embodiments, the Brix value of the malt is between about 20 and about 50, between about 25 and about 50, between about 30 and about 50, between about 35 and about 50, between about 40 and about 50 or between about 45 and about 50.

In certain embodiments, the Brix value of the malt is between 20 and 50, between 25 and 50, between 30 and 50, between 35 and 50, between 40 and about 50 or between 45 and 50. In certain embodiments, the refractive index value of the malt is between 1.36 and 1.45, between 1.37 and 1.45, between 1.38 and 1.45, between 1.39 and 1.45, between 1.40 and about 1.45 or between 1.41 and 1.45.

### Optional Filtration Step

Once the refractive index or Brix attain a value of at least 20, the mash dispersion can be optionally filtered to remove turbidity while retaining surface activity.

In certain embodiments, protein can be removed either by ultra-filtration or by flocculation with salt, sugar, pH, heat, filtration, followed by a crude filtration to remove cell wall materials.

In certain embodiments, higher Mw fractions can be removed using ultrafiltration.

In certain embodiments, sugars, salts, water (i.e. concentrate) are removed using nanofiltration.

In certain embodiments, the mash is not filtered.

In certain embodiments, the mash further contains whole seed proteins and/or cellulose.

### Emulsification/Dispersion Step

In certain embodiments, flavor oil is then added to the mash filtrate, mixed in a Silverson mixer followed by high pressure emulsification into a holding tank.

In certain embodiments, an emulsion or dispersion can be generated using a static mixer, rotor stator homogenizer, high shear mixer, colloid mill, or high-pressure homogenizer

In certain embodiments, inherent oils increase the viscosity of the oil phase. In certain embodiments, elongational stress such as applied by a static mixer may be used to reduce oil droplet size. In certain embodiments, the flour's inherent cellulose may be modified in size during the dispersion step for stabilizing the emulsion and/or beneficially impacting subsequent performance.

In certain embodiments, active components of whole grain (fatty acids, phospholipid and proteins) contribute to emulsification of the active ingredients thus alleviating the need for surfactant additives. These components are stripped away from processed starch such as maltodextrin from refined starch.

### Drying Step

The flavor oil emulsion can be then spray dried according to methods that are well known in the art to produce a food powder encapsulate.

In certain embodiments, the size of the cellulose alters the size of atomized droplets.

In certain embodiments, a centrifugal wheel is employed that prevents the clogging nozzles with particulates. In certain embodiments, high viscosity can be accommodated by using an ultrasonic nozzle.

### Exemplary Encapsulates

Exemplary encapsulates manufactured with the aforementioned encapsulation process include a sweet vanilla mint powder that provides a rounded flavor profile and enhanced stability in breath mints, an organic peppermint hot chocolate concept or tortilla chips with a powdered lime flavor with a high oxidative stability and handling benefits in production.

Powders and granules developed from the disclosed encapsulation system are useful for preserving and delivering flavors within preparations of desserts, cakes and biscuits mixes, snacks, tea bags, loose tea, coffee, instant beverages, confectioneries and blender prepared nutritional beverages. The disclosed encapsulates are particularly useful to prepare natural or clean label food products as well as fragrance powders or granules.

In certain embodiments, the encapsulates comprise prebiotics, soluble fiber oligosaccharides, human milk oligosaccharides, probiotics (e.g. lactobacilli (Lactobacillus acidophilus and Lactobacillus GG), bifidobacteria (Bifidobacterium bifidus) and some yeasts (like Saccharomyces boulardii) or carotenoids (e.g., beta carotene or lycopene).

### Alternative Encapsulation Methods

The encapsulate can be produced by any of range of technologies known in the art including spray drying, low temperature spray drying, multistage spray drying, spray granulation, spray agglomeration, drying of starch-coated beadlets, fluidized bed drying, fluidized bed coating, melt injection, melt extrusion, drum drying, freeze drying, vacuum drying, prilling, belt drying, refractance window drying, mylar belt radiative drying. Or combinations of encapsulation technologies such as spray drying followed by agglomeration, powder compaction or extrusion. These microencapsulation methods are further summarized in publications such as Sagalowicz and Leser “Delivery systems for liquid food products” (Current Opinion in Colloid &amp; Interface Science 15 (2010) 61-72). Another reference that discusses microencapsulation processes is the book edited by Zuidam and Nedovic “Encapsulation Technologies for Active Food Ingredients and Food Processing” (2008).

In certain embodiments, the encapsulation process disclosed herein can be applied to natural flavors, natural colors, coloring foodstuffs (such as turmeric), natural extracts (like flax seed oil), spice extracts. fruit juices, cannabidiol (CBD) or poly unsaturated fatty acids from fish or algae oils.

In certain embodiments, the aforementioned malting and mashing processes can be combined directly with a flavor encapsulation process. Previously, starches were mashed with enzymes to make maltodextrins which were dried to create an intermediate ingredient. Combining the processes can be advantageous because it removes an extra drying step making the process simpler with a clean label appeal.

In certain other embodiments, the malting and mashing is a two-step process in which, for example, grains are mashed, dried and the intermediate is reconstituted and combined with the active ingredient, such as a natural flavor, and dried.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict between the present explicit disclosure and a document incorporated by reference, the present explicit disclosure shall be the operative disclosure.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### EXAMPLES

### Example I: A Starch and Flour Based Flavor Encapsulation System

A starch and flour-based carbohydrate matrix is prepared comprising: 0% modified starch, maltodextrin, starch hydrolysate or isolated dextrin; 50-100% native starch or flour such as rice flour, rice starch, tapioca flour, corn starch, starches and flours; and 0.5 to 30% of a natural source of enzymes including malted rice, malted barley, malted corn, malted millet or malted buckwheat. The starch/flour and the source of enzymes are then combined and conditioned in water to achieve a viscosity of less than 1 Pascal second or 1000 centipoise with a solids loading greater than 20%.

In this example, 80 g of malted barley flour was dispersed into 600 g water and brought to 65° C. with constant stirring. Then 320 g of tapioca flour was added under constant stirring and the temperature increased to 70° C. for mashing. Additional instant tapioca flour can be added during mashing to increase the total solids level, as high solids levels are known to favor flavor retention during the later drying stage. The malted grain was then conditioned along with the starch/flour to reduce the viscosity of the combined mixture. The conditioning occurred within a vessel with mixing at temperatures between 25° C. and 70° C. that are conducive for alpha amylase to convert starches into less viscous components. Optional components that may be added as needed include emulsifiers, pH regulators, and/or antioxidants.

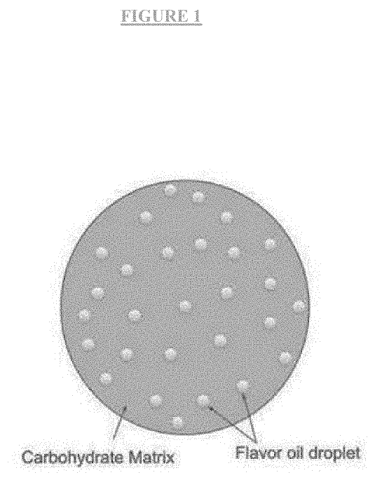

A flavor oil was then added to the starch and flour-based carbohydrate matrix and emulsified to form a multitude of droplets that were enrobed within the carrier as shown in FIG. 1. In this example, the emulsified mixture was then further processed to form a solid encapsulate, The oil was d-limonene and the drying was convective drying of a thin film.

### Example II: Preparation of Mint Flavor Powder

Rice seed (Oryza sativa) L. indica is steeped, germinated and kilned at 30° C./24 hours, 25° C./5 days and 50° C./4 hours respectively as described in Usansa U, Sampong N, Wanapu C, Boonkerd N, Teaumroong N. The Influences of Steeping Duration and Temperature on the α- and β-amylase Activities of Six Thai Rice Malt Cultivars (Oryza sativa L. indica). Journal of the Institute of Brewing. 2009; 115: 140-147. The grain was dehulled and ground to create a flour using a kitchenaid mixer and the grain milling attachment. In some examples, the Malted Sweet Rice Flour is obtained from Eckert Malting and Brewing, Chico Calif.

A mixture of 65 parts water and 35 parts rice flour was prepared by first bringing the water to 70 C under stirring. Rice flour was added in 4 equal increments each separated by 10 minutes in order to allow time for starch gelatinization and mashing while maintaining viscosity below 1000 centipoise. The mixture was processed using a high shear mixer (Silverson LMA-5) to break down residual particles and mashed for an additional 60 minutes at 70° C. At this stage the refractive index was measured using a Atago PAL-1 refractometer and found to be 32 brix suggesting that starches within the flour had been significantly solubilized. The preparation was tested for viscosity using a Brookfield DV1 viscometer with spindle LV3 a speed 50 and found to be 160 mPa·s, a level one skilled in the art would consider amenable for spray dry processing. The preparation was cooled to 60° C. where the 100 parts of the flour/water suspension were combined with 3.9 parts mint oil (Natural Flavors Co) and mixed under high shear to form an emulsion. The emulsion was transferred by peristaltic pump through a flexible hose to the atomizing nozzle of a lab spray dryer (Bowen 36″ Lab Dryer with 2 fluid atomizing nozzle 100 psi, inlet temperature 330 F outlet 220° F.) and dried to create a mint flavor powder.

### Example III: Traditional Rice Starch and Flour Cannot be Used as a Spray Drying Carrier

300 g of Vihn Thaun brand rice starch was combined with 700 g of water with a Bellini BMKM510CL food processor. The preparation was heated to a 100° C. setpoint with stirring to gelatinize the starch. Prior to reaching the setpoint, at 75° C., the mixture took on a viscous dough-like consistency and proceeded to spin within the food processor releasing from the walls. This demonstrates that simply cooking rice starch does not lead to compositions amenable for spray processing. Traditional rice starch and flour cannot be used as a spray drying carrier.

### Example IV: 15% Lime Oil and Sprouted Rice Flour

A mixture of 1300 g water and 700 g organic sprouted brown rice flour (Everspring Farms product code BRLOSW00) was prepared by first bringing the water to 70 C under stirring. Rice flour was added in 3 equal increments along with 200 microliters of BAN480L non-GMO derived amylase enzyme (from Novozymes A/S Copenhagen, Denmark.) The mixture was processed at 10,000 rpm for 10 minutes using a high shear mixer (Silverson LMA-5) to break down residual rice particles. An addition 400 microliters of amylase enzyme was added and the mixture was mashed for 30 minutes at 70 C. At this stage the refractive index was measured using a Atago PAL-1 refractometer and found to be 29.2 brix suggesting that starches within the flour had been significantly solubilized. The preparation was tested for viscosity using a Brookfield DV1 viscometer with spindle LV3 a speed 50 and found to be 235 mPa·s. A measure of 139 g of cold pressed lime oil (LorrAnn's Natural Lime Oil item 0110-0800) was added to the preparation under stirring and then mixed at high shear to form an emulsion. The lime oil and rice flour emulsion was fed by pump to an Anhydro Lab spray dryer using a bottom vertical nozzle. Inlet temperature was 160 C and outlet was 90 C. Dry powder was collected having a potent lime flavor character.

### Example V: 17% Beet Juice Solids and Sprouted Rice Flour

A mixture of 2000 g water and 1000 g organic sprouted brown rice flour (One Degree Organic Foods, Sprouted Brown Rice Flour) was prepared by first bringing the water to 70 C under stirring. Rice flour was added in 3 equal increments along with 300 microliters of BAN480L non-GMO derived amylase enzyme (from Novozymes A/S Copenhagen, Denmark) at each addition. The mixture was processed at 10,000 rpm for 10 minutes using a high shear mixer (Silverson LMA-5) to break down residual rice particles. An addition 400 microliters of amylase enzyme was added and the mixture was mashed for 30 minutes at 70 C. At this stage the refractive index was measured using a Atago PAL-1 refractometer and found to be 28.1 brix suggesting that starches within the flour had been significantly solubilized. The preparation was tested for viscosity using a Brookfield DV1 viscometer with spindle LV3 a speed 50 and found to be 352 mPa·s. A measure of 472 g liquid beet juice concentrate (Brownwood Acres Eastport Mich., UPC:18303000504) was mixed into the flour water dispersion. The beet juice dispersion was transferred by peristaltic pump through a flexible hose to the atomizing nozzle of a lab spray dryer (Bowen 36″ Lab Dryer with 2 fluid atomizing nozzle 100 psi, inlet temperature 330 F outlet 220 F) and dried to create a purple coloring powder.

### Example VI: Tableted Breath Mints

The encapsulated mint powder of Example II was combined at a 9:1 ratio with sucrose. This powder blend can be compressed into 5 mm circular tablets using a WellieSTR 1 manual press. The finished tablets suitably serve as clean label breath mints.

### Example VII: Chocolate Instant Beverage

A hot chocolate mix was created by dry blending 1100 g organic cane sugar, 1000 g organic brown sugar, 450 g of organic cocoa powder, 450 g of organic dark chocolate chips, 46 g of sea salt and 30 g of peppermint oil powder from Example II. All ingredients except the peppermint powder were 365 brand purchased from Wholefoods supermarket. 15 g portions of the blended cocoa mix were distributed into envelops to create single serving clean label mint hot chocolate beverage products.

### Example VIII: Lime Flavored Tortilla Chips

Organic multigrain tortilla chips (product sku 77890 36023) were attained from Wegmans grocery store. A measure of 1.5 g of powdered lime flavor from Example III was dusted onto 500 g of the tortilla chips and dispersed by shaking the dusted chips with in a bag to produce a lime-flavored tortilla chip product.

### Example IX: Instant Red Raspberry Limeaid Beverage

An instant beverage powder blend is created with the following components
57 g citric acid
911 g sugar
12 g raspberry concentrate powder (Natures Flavors lot 190102012)
3 g lime flavor powder of Example IV
17 g beet juice powder of Example V
87.5 g of this powder is reconstituted with water to a volume of 1 liter to create a
Red Raspberry Limeade beverage.

### Example X: Limonene Emulsions with Natural Surfactants

A mixture of 60 parts water and 40 parts rice flour was prepared by first bringing the water to 70° C. under stirring. Malted sweet rice flour, Eckert Malting, Chico, Calif. was added in 2 equal increments each separated by 10 minutes in order to allow time for starch gelatinization and mashing while maintaining viscosity below 1000 centipoise. The mixture was processed using a high shear mixer (Silverson LMA-5) to break down residual particles and mashed for an additional 60 minutes at 70° C. At this stage, the refractive index was measured using a Atago PAL-1 refractometer and found to be approximately 36 brix, suggesting that components within the flour had been significantly solubilized.

Next, the preparation was cooled to 60° C., where limonene oil was added to achieve a limonene to flour ratio of 20:80. The preparation was mixed under high shear to form an emulsion using a Silverson LMA-5 for 10 minutes. A small aliquot of the emulsion was diluted 10× with water, placed on a glass slide with coverslip and photographed at 400× using a LW Scientific i5 microscope. A field of view was selected with more than 100 droplets. The average size of the four largest droplets is reported in Table II below as the max droplet size range.

TABLE II

| | Max droplet |
|---|---|
| Emulsion type | size range |
| Rice flour with 20% Limonene | 10-15 microns |
| Rice flour with 20% Limonene with Mustard flour | 10-15 microns |
| Rice flour with 20% Limonene with Pea Protein | 7-14 microns |
| Riceflour with 20% Limonene and Lecithin | 10-16 microns |
| Pea sprouts with 20% Limonene | 4-10 microns |

Separately, a mixture of 60 parts water, 40 parts rice flour and 10 parts limonene was prepared as above. An additional 0.5 parts of McCormick brand mustard flour was added as a natural emulsifier and homogenized as above. An aliquot of the emulsion was diluted and characterized by microscopy.

Separately, a mixture of 60 parts water, 40 parts rice flour and 10 parts limonene was prepared as above. An additional 0.5 parts of Vitessence Pulse 1803 pea protein concentrate (Ingredion Inc.) was added as a natural emulsifier and homogenized as above. An aliquot of the emulsion was diluted and characterized by microscopy.

Separately, a mixture of 60 parts water, 40 parts rice flour and 10 parts limonene was prepared as above. An additional 1 part of soy lecithin (CVS Health brand) was added as a natural emulsifier. The lecithin was pre-dissolved into the limonene prior to being added to the rice flour/water preparation and then homogenized. An aliquot of the emulsion was diluted and characterized by microscopy.

Separately, organic whole dried green peas (Food to Live brand, Lexunder Inc. Brooklyn N.Y.) were steeped in 20° C. water for 12 hours. The peas were germinated in a sprouting vessel and washed every 12 hours for 3 days. The pea sprouts were then combined with water to produce a slurry with 20% solids and mashed for 90 minutes at 70° C. The brix value increased with mashing time and reached a maximum of 11.4 brix. The preparation was cooled to 60° C., where limonene oil was added to achieve a limonene to pea solids ratio of 20:80.

In some examples, the emulsions are spray dried to form powders. In other examples, the pea sprouts are spray dried to form a flour.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. An encapsulate comprising a clean label carrier dispersed with an active ingredient.

2. The encapsulate of claim 1, wherein the clean label carrier does not contain processed starch.

3. The encapsulate of claim 2, wherein the processed starch comprises a chemically modified starch, maltodextrin from refined starch, tapioca, potato or cereal syrup solids.

4. The encapsulate of claim 1, wherein the clean label carrier does not contain a gum extracted from plants or a microorganism, artificial flavors, artificial sweeteners or artificial preservatives.

5. The encapsulate of claim 4, wherein the gum comprises gum arabic, locust bean gum, gum ghatti, gum guaiacum, gum guar, or gum karaya.

6. The encapsulate of claim 1, wherein the clean label carrier is mashed to a refractive index of more than about 1.36 when measured at 25° C.

7. The encapsulate of claim 1, wherein the active ingredient comprises a natural extract, a fruit or vegetable juice concentrate, a natural flavor, a pharmaceutical, a cannabidiol (CBD), an essence, a natural color, a juice extract, a biosynthetic color, cheese, nutraceutical and/or nutritional oil or any combination thereof.

8. The encapsulate of claim 1, wherein the active ingredient comprises a natural flavor.

9. The encapsulate of claim 8, wherein the natural flavor comprises a mint or lime oil.

10. The encapsulate of claim 1, wherein the active ingredient comprises a natural color.

11. The encapsulate of claim 10, wherein the natural color comprises beet juice.

12. The encapsulate of claim 1, wherein the clean label carrier comprises a malt of at least partially germinated whole grain seed, pulses, legumes, false grains or flour derived therefrom.

13. The encapsulate of claim 1, wherein the clean label carrier comprises a malt of at least partially germinated legumes comprising chickpea, yellow pea, lentil, mung bean, green pea or flour derived therefrom.

14. The encapsulate of claim 1, wherein the clean label carrier comprises a malt of amaranth, barley, buckwheat, corn, millet, oats, quinoa, rice, rye, sorghum, teff, triticale, wheat and wild rice.

15. The encapsulate of claim 1, wherein the clean label carrier comprises a malt of rice flour.

16. The encapsulate of claim 12, wherein the malt comprises activated endogenous glycosidases comprising alpha-amylases.

17. The encapsulate of claim 12, wherein the clean label carrier further comprises carbohydrate sources from green bananas, peas, beans, roots, potato, tapioca, tapioca starch, rice starch, purple sweet potato, sweet potato and/or yam

18. The encapsulate of claim 12, wherein the malt further comprises added exogenous glycosidases.

19. The encapsulate of claim 12, wherein the clean label carrier is encapsulated by spray drying, low temperature spray drying, multistage spray drying, spray granulation, spray agglomeration, drying of starch-coated beadlets, fluidized bed drying, fluidized bed coating, melt injection, melt extrusion, drum drying, freeze drying, vacuum drying, prilling, belt drying, refractance window drying, mylar belt radiative drying or any combination thereof.

20. A foodstuff comprising an encapsulate of claim 19.

21. The foodstuff of claim 20, wherein the foodstuff is a snack, a confection, dry beverage, dry doughs such as cake, cookie or bread mixes, stock cubes, crackers or tortilla, bakery mix, pancake mix, breakfast cereals, bars, protein smoothies, stuffing, coffee/tea nutritional supplements, instant sauce mixes, spice rubs and marinades, or meat and meat-alternatives.

22. A method of making an encapsulate comprising:
mashing a malt of a whole grain seed, pulses, legumes, false grains or flour derived thereof, wherein the mash has a Brix value of at least 20;
optionally filtering the mash;
dispersing the mash with an active ingredient; and
encapsulating the mash dispersion.

23. The method of claim 22, wherein the ratio of the Brix value relative to the amount of flour solids present is at least 0.5:1.

24. The method of claim 22, wherein the encapsulated mash dispersion is agglomerated, roller compacted, extrusion pelletized or coated.

25. The method of claim 22, wherein the encapsulate does not contain processed starch, a gum extracted from plants, artificial flavors, artificial sweeteners or artificial preservatives.

26. The method of claim 22, wherein the active ingredient comprises a natural extract, a fruit or vegetable juice concentrate, a natural flavor, a pharmaceutical, a cannabidiol (CBD), an essence, a natural color, a juice extract, a biosynthetic color, cheese, nutraceutical and/or nutritional oil or any combination thereof.

27. The method of claim 22, wherein the malt comprises a malt of rice flour.
